(19) United States
(12) Patent Application Publication
(10) Pub. No.: US 2005/0192558 A1
(43) Pub. Date: Sep. 1, 2005

(54) Peripheral access venous cannula with infusion side holes and embedded reinforcement

(75) Inventors: Steven Bernard, Andover, MN (US); Sean Patrick Skubitz, Shoreview, MN (US)

Correspondence Address: NIXON & VANDERHYE, PC, 901 NORTH GLEBE ROAD, 11TH FLOOR, ARLINGTON, VA, 22203, US

(73) Assignee: CHF Solutions, Inc., Brooklyn Park, MN (US)

(21) Appl. No.: 10/787,742

(22) Filed: Feb. 27, 2004

(51) Int. Cl. A61M 25/00

(52) U.S. Cl. 604/525

(57) ABSTRACT

A peripherally inserted catheter assembly having multiple side holes to be inserted in a peripheral vein and maneuvered upward through the vascular system to access the reservoir of blood beyond and between the venous flappers for continuous blood withdrawal and treatment.

### RELATED APPLICATION

This application is a continuation-in-part application (CIP) of and claims the benefit of the priority of U.S. patent application Ser. No. 10/______(Attny. Ref. No. 3659-74), (now U.S. Pat. No. ______) entitled “Method And Apparatus For Ultrafiltration Utilizing A Peripheral Access Dual Lumen Venous Cannula” and filed Dec. 2, 2003, the entirety of which is incorporated by reference.

### BACKGROUND OF THE INVENTION

This invention relates to the extracorporeal treatment of blood with a peripheral access cannula. The invention may be applied to blood access for Renal Replacement Therapy, treatments using an artificial kidney, and for the treatment of congestive heart failure (CHF).

Congestive Heart Failure (CHF) patients can benefit from fluid removal by ultrafiltration of blood. CHF patients tend to have functional kidneys, but suffer from fluid overload due to CHF. The kidneys of CHF patients are generally healthy, but are not fully functioning due to the failing heart and low blood pressure. Because their kidneys are not fully functioning, fluids build up in the patient (fluid overload) contributes to the stress on the already failing heart. This added stress can cause the heart to further deteriorate. Even though they do not remove sufficient fluid, kidneys of a CHF patient often do produce sufficient urine to remove toxic solutes from the blood. This small amount of urine that is produced is not sufficient to avoid fluid overload of the patient.

With the increasing prevalence of decompensated CHF and the increased cost of hospital admission and even more so of an intensive core unit (ICU) treatment, a strong need has emerged for a blood treatment system that will allow fluid removal in the non critical care setting, and that is simple and safe so that it could be used in the outpatient setting, doctor's offices, emergency rooms (ER) and general hospital floors. Such a system would be acceptable if access to venous blood was established via a peripheral vein in the patient's arm or other peripheral vascular site on the patient. Accessing blood through a peripheral vein in the arm is advantageous. The peripheral veins are close to skin, easy to identify and can be readily accessed. Physicians and nurses are trained to insert needles and catheters in the peripheral veins of an arm. Venopunctures are easy to monitor for infiltration of fluid and thrombosis and the control of infection is simpler than with central catheters. The potential loss of a peripheral vein to thrombosis is less critical.

Ultrafiltration (a mode of Renal Replacement Therapy) is useful for removal of excess fluid from a patient, especially in CHF patients whose kidneys are not fully functioning due to CHF but are otherwise generally healthy. An ultrafiltration technique has been recently developed that relies on peripheral vein access. This ultrafiltration technique is described in U.S. Pat. No. 6,533,747 entitled “Extracorporeal Circuit for Peripheral Vein Fluid Removal”, and in U.S. Pat. No. ______ (now pending U.S. patent application Ser. No. 09/618,759, filed Jul. 18, 2000 (atty. ref. 3659-10)), entitled “Method and Apparatus for Peripheral Vein Fluid Removal in Heart Failure”. The volume of blood that can be drawn from a peripheral vein is substantially less than that which can be drawn from a central access vein. Nevertheless, the relatively-small volume of blood removed from peripheral veins has been found sufficient for ultrafiltration for many CHF patients suffering from fluid overload.

Some patients have peripheral veins that do not allow for removal of enough blood for ultrafiltration using a short needle withdrawal catheter. The peripheral veins in these patients have insufficient blood flow (antegrade flow) to prevent collapse of the vessel while blood is withdrawn through a short needle catheter. The antegrade blood flow in a peripheral vein may be insufficient by itself for ultrafiltration. Moreover, retrograde flow of blood into a short needle catheter is prevented by the flapper veins in peripheral veins. Retrograde flow is blood flow opposite to the normal flow direction (antegrade) in the vein.

The natural purpose of venous flapper valves is to prevent retrograde blood flow when a person moves and thereby applies inertia and centrifugal forces to the blood in the veins. The valves also prevent pooling of blood at the lower extremities, e.g., hands and feet, due to the force of gravity. These venous flapper valves, which appear to work quite well even in CHF patients, prevent retrograde flow of blood into a short peripheral catheter inserted into the arm of a CHF patient.

Short needle catheters do not extend beyond the flappers in the peripheral vein. It is believed that the venous flappers are a principal reason why retrograde flow is prevented when a peripheral catheter applies a local negative pressure in a peripheral vein. Accordingly, there is a need to overcome or circumvent the natural venous flappers. By circumventing these flappers, a peripherally inserted catheter should be able to create a sufficient negative pressure to cause retrograde blood flow and, thus, increase the blood flow through a catheter for ultrafiltration treatment without collapsing the vein. A single lumen long catheter for ultrafiltration is disclosed in U.S. Pat. No. 6,685,664, entitled “Method And Apparatus For Ultrafiltration Utilizing A Long Peripheral Access Venous Cannula For Blood Withdrawal.”

### SUMMARY OF INVENTION

A solution to these shortcomings of existing catheters has been discovered. The catheter design disclosed herein circumvents the venous flappers to provide adequate blood supply for ultrafiltration treatment. The result is a dual lumen catheter with a withdrawal lumen that uses its distal tip and side hole inlets for blood withdrawal from a peripheral vein. By withdrawing blood from an inlet at the distal end and side holes, the catheter is able to draw from a large pool of blood by accessing the retrograde blood available in the portion of the vein downstream of the veneous flappers and the blood between venous flappers in upstream portions of the vein. Moreover, the catheter is reinforced at the side hole locations to prevent kinking as the long catheter slides into the vein. The catheter produces acceptable operational negative pressures for blood withdrawal by optimizing the cross sectional areas of the internal flow lumen(s) throughout the catheter length.

In one embodiment, the invention is a catheter for withdrawing blood through a peripheral vein of a patient, said catheter comprising: a withdrawal lumen having an insertable length of at least ten centimeters (cm); a distal tip of the withdrawal lumen at a distal end of the insertable length of the withdrawal lumen and having a blood receiving opening at the distal end, and a plurality of sidewall apertures in a sidewall of the catheter, wherein the sidewall apertures receive blood into the withdrawal lumen.

### BRIEF DESCRIPTION OF THE DRAWINGS

An embodiments of the invention are illustrated in the attached drawings that are described as follows:

FIG. 1 is an illustration of a transverse section of a peripheral vein showing open and closed vein flapper valves and a dual lumen catheter inserted in the vein.

FIG. 2 is a side view of a portion of a dual lumen catheter showing the side holes in the withdrawal lumen and stiffer durometer sections.

FIG. 3 is a top view as indicated by line 3-3 in FIG. 2 of a portion of the dual lumen catheter showing a discharge opening for the infusion lumen and stiffer durometer sections at the side holes.

FIG. 4 is a partial cross section view of a portion of the dual lumen catheter showing in cross-section a reinforcement coil embedded in the catheter.

FIG. 5 is a side view with a portion cut away of the dual lumen catheter showing a reinforcing coil, wherein the coil of FIG. 4 is not shown in cross-section.

FIG. 6 is a side view of a portion of a single lumen catheter showing side hole inlets and stiffer durometer sections.

FIG. 7 is a side view with a portion cut away of the single lumen catheter showing the coil reinforcement in cross-section.

### DETAILED DESCRIPTION OF THE INVENTION

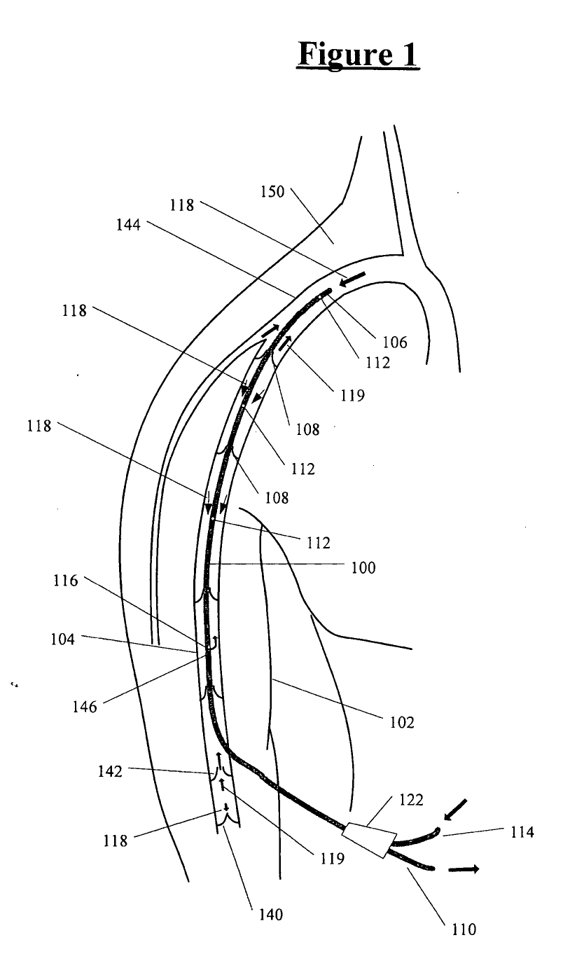

FIG. 1 shows a dual lumen middle length peripheral access venous blood cannula 100, commonly called a Peripherally Inserted Central Catheter (PICC), which may be applied to withdraw and infuse blood for an extracorporeal blood circuit of, for example, an ultrafiltration system (not shown). The cannula is inserted into a surface peripheral vein 104 of the arm 102 of a patient. The cannula includes a distal tip 106 that is advanced along through vein and past the venous flappers 108 in the vein. The distal tip includes an aperture that withdraws blood from the vein into a withdrawal lumen 110 that extends the length of the cannula. In addition, withdrawal side holes 112 in the cannula withdraw blood from the vein into the withdrawal lumen. An infusion lumen 114 extends from the proximal end of the PICC, through the vein skin insertion location on the arm and a short distance, e.g., 5-10 centimeters, into the vein to an infusion port 116. The PICC may be a single use disposable cannula used for the ultrafiltration of blood to treat fluid-overload in patients. The PICC is intended to be used in a non-ICU medical environment and without surgery.

As shown in FIG. 1, a slight negative pressure in the catheter 100 causes blood flow in the vein to enter the tip 106 and enter the side holes 112 arranged at various locations along the length of the withdrawal lumen 110 (including in the vicinity of the infusion holes 116, every two centimeters distally of the infusion holes, and within two centimeters of the distal tip). The blood flow drawn into the lumen 110 includes antegrade flow 119 and retrograde flow 118. Air-tight connectors (not shown) are incorporated in the catheter assembly to overcome the untoward effects of negative pressure in blood withdrawal.

During insertion, the insertable portion of the cannula 100 is advanced through the venous tree from the peripheral vein 104 and into the shoulder of the patient. The tip 106 is downstream (with respect to the normal veneous blood flow) of the flappers 108 in the peripheral veins. Because of the position of the catheter tip upstream of all of the flappers, the flappers do not prevent retrograde 118 blood flow into the withdrawal lumen 110. The flappers close 140 to block retrograde flow 118, and open 142 to pass antegrade flow 119. With respect to the withdrawal of blood, the retrograde flow supplements the antegrade flow so that there is a sufficient flow of withdrawn blood into the withdrawal lumen 110 and to maintain sufficient pressure in the vein to prevent vein collapse during withdrawal.

The tip 106 is positioned at the shoulder 150 and in an axillary vein, subclavian vein or vena cava, (collectively 144) which are in the venous tree that includes the peripheral vein receiving the cannula. At these locations, the tip and distal side holes 112 may draw from the large volume of blood available through the central venous passages. The retrograde flow 118 into the distal tip draws blood through the vein from the central body venous blood supply. Similarly, retrograde flow may also occur at other withdrawal side holes 112 (proximal to the tip), although the venous flappers 108 upstream of these side holes limit the amount of retrograde flow into those side holes. By drawing blood using retrograde and antegrade flows, the PICC catheter 100 provides blood to an extracorporeal circuit at a flow rate greater than the rate that could be withdrawn using antegrade blood flow alone, such as could be obtained by using short phlebotomy withdrawal needles. Accordingly, a PICC catheter has the double benefit of the safety, ease and comfort of peripheral vein access, and the high withdrawal flow rate available when using retrograde venous flows.

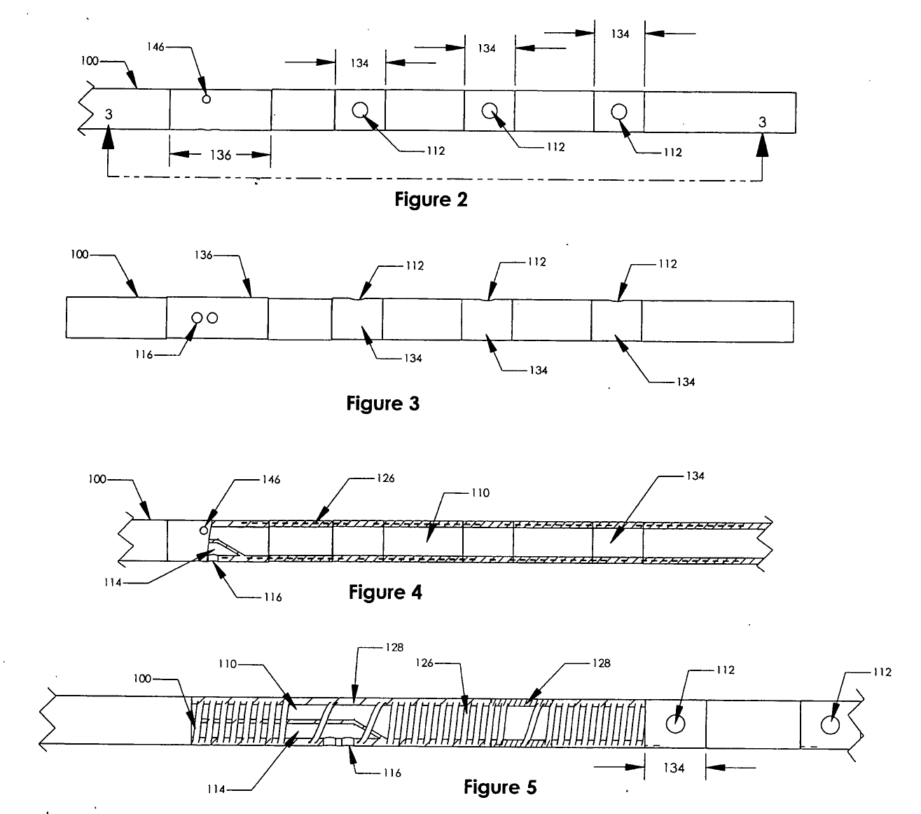

FIGS. 2 to 5 show a portion of the dual lumen catheter 100 including a distal end of the infusion lumen 114 and a section of the withdrawal lumen distal to the infusion lumen vein. The infusion lumen and withdrawal lumen extend side by side from the hub 122 of the catheter to the infusion port 116, where the infusion lumen ends. Beyond the infusion port 116, the withdrawal lumen 110 continues alone to the tip 106. The withdrawal lumen also includes withdrawal side hole apertures 146 in a sidewall of the catheter near the infusion ports 116. These side holes 146, along with the other side holes 112, draw blood from the vein into the withdrawal lumen. These side holes 146 will also ingest air if the catheter is inadvertently pulled so far out of the skin that the infusion port 116 is exposed to air. A bubble detector in the blood circuit senses the ingested air in the blood passage of the blood circuit, and stops both the infusion of blood out of the catheter and the withdrawal of air into the catheter.

The sidewall openings (also referred to as apertures) 112, 146 are supported by a continuous reinforcing member 126, e.g. embedded coil or braided fibers, embedded in the sidewall of the cannula. The reinforcing member 126 provides support to the catheter outer wall improving column strength, radial strength, kink resistance, and crush resistance, especially where the infusion lumen terminates. Additionally, the presence of the reinforcing member allows the catheter to be designed with a thin wall that has a relatively-large cross sectional area for the dual lumens while minimizing the catheter outside diameter. The PICC wall thickness may be 0.006 of an inch to 0.010 inch, for example.

The durometer of the PICC catheter tubing over its insertable length is preferably between 90 to 115 Shore A, so as to prevent lumen pinching. Multiple tubing durometers, fillers, and material formulations may also be used to create reliable withdrawal and infusion lumens. The catheter durometer may be increased to 55 to 75 Shore D, or made substantially ridged in the area of the infusion opening 116 or withdrawal side holes 112 and the area within 1.5 cm proximally and distally of the openings 116, 112. The length of each section 134 with increased durometer or ridges may be two centimeters. An overall section 136 of the catheter aligned with the infusion ports 116 of preferably no more than three cm may be ridged or semi-ridged tubing should prevent tube kinking at the openings 112, 116, 146 and to promote easy insertion of the catheter. Other reinforcing members 126 materials are embedded coils, braids, durometer transitions, or combinations thereof, polymeric or metallic.

The withdrawal inlet side holes 112 may be spaced along the insertable length of the withdrawal lumen at regular intervals, e.g., 2 cm, that correspond to the typical distance between flapper valves in a peripheral vein. The number and diameter of side holes 112, 116 and 146 should be limited to maintain catheter integrity and minimize areas for clots to form. The relative short term duration, e.g. 4 to 24 hours in duration, and low frequency, e.g., once or twice in a week, of ultrafiltration therapy enables the side-holes to be used without a significant risk of catheter clotting or thrombosis.

The number of side holes 112, 146 and their diameter may be limited to preserve the catheters ability to be fully flushed down the length of the catheter. If the holes are too large or there are an excess number then the flush “locking solution” may leak out of the catheter over time. Also, if there are too many side holes, the catheter may not draw sufficiently throughout its length to keep the entire length patent. Furthermore, the orientation of the withdrawal and infusion side holes can be positioned in several configurations along the length of the cannula including linear, alternating, spiraled, or a combination of these arrangements. Moreover, there may be several side holes arranged near the distal tip to increase the capacity of the withdrawal lumen to receive retrograde flow from a central veneous blood supply.

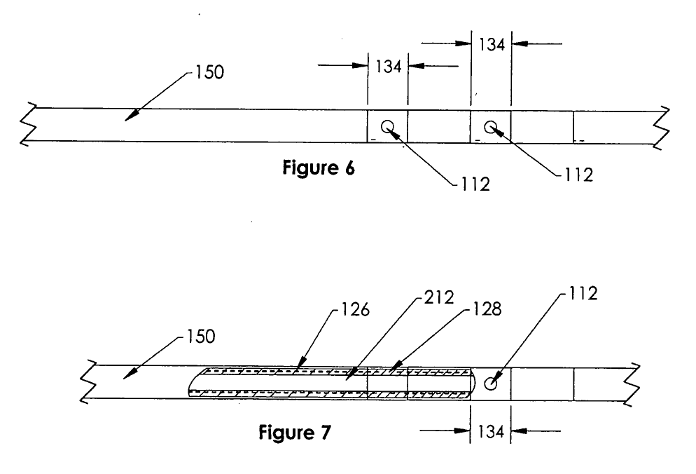

FIGS. 6 and 7 are diagrams of a portion of a single lumen catheter 150 showing a coil reinforcing member 126 in the wall of the catheter. The reinforcing member 126 may be placed in a portion of the catheter between the proximal and distal regions, e.g., surrounding the sidewall openings, or along the entire catheter length. The reinforcing member 126 may be a braid or wire coil. The braid pic rate or the coil pitch rate of the reinforcing member may be continuous or vary throughout the length of the catheter to change properties and provide gaps 128 in the catheter wall at the infusion opening 116 and side holes 112 in the withdrawal lumen without severing the braid or coil.

The present invention has been described in terms of a particular embodiment(s). The invention is not limited to the disclosed embodiment(s). The scope of the present invention is defined by the spirit and scope of the claims that follow.

1. A catheter for withdrawing blood through a peripheral vein of a patient, said catheter comprising:
a withdrawal lumen having an insertable length of at least ten centimeters (cm);
a distal tip of the withdrawal lumen at a distal end of the insertable length of the withdrawal lumen and having a blood receiving opening at the distal end, and
a plurality of sidewall apertures in a sidewall of the catheter, wherein the sidewall apertures receive blood into the withdrawal lumen.

2. A catheter as in 1 further comprising a stiff catheter section aligned with the sidewall apertures, wherein said stiff catheter section has a stiffness at least ten percent greater than a stiffness of the catheter beyond the stiff catheter section.

3. A catheter as in claim 2 wherein the stiff catheter section has a length of at least 0.5 cm and a distance of at least two centimeters exists between the sidewall apertures.

4. A catheter as in 1 further comprising a coil reinforcing at least a portion of the insertable length of the catheter.

5. A catheter as in claim 1 wherein said sidewall apertures are separated from each other by at least two centimeters.

6. A catheter as in claim 1 further comprising an infusion lumen adjacent to the withdrawal lumen and extending partially along the insertable length of the catheter, wherein said infusion lumen has a distal port

7. A catheter as in claim 1 wherein the catheter has a substantially uniform cross-sectional periphery along the insertable length.

8. A catheter as in claim 1 wherein said catheter has a constant outside diameter section along its insertable length up to the distal tip.

9. A catheter as in claim 1 wherein the catheter is formed of 90 to 110 Shore A polyurethane.

10. A catheter as in claim 1 where the catheter is a dual lumen catheter.

11. A catheter as in claim 1 wherein the catheter is a single lumen catheter

12. A catheter as in claim 1 wherein the sidewall aperture includes at least one distal sidewall aperture within two centimeters of the distal tip and the at least one distal sidewall aperture has an opening cross sectional area at least as great as a cross section of withdrawal lumen adjacent the distal opening.

13. A catheter as in claim 1 wherein each fluid withdrawal lumen side hole openings has a cross sectional area of less than a cross section of the withdrawal lumen adjacent the opening.

14. A dual lumen catheter as in claim 13 further comprising a tube reinforcing member in a section of tube wall at least adjacent to the side wall openings.
